(19) United States
(12) Patent Application Publication
(10) Pub. No.: US 2024/0199085 A1
(43) Pub. Date: Jun. 20, 2024

(54) VEHICLE CONTROL APPARATUS AND METHOD THEREOF

(71) Applicants: HYUNDAI MOTOR COMPANY, Seoul (KR); KIA CORPORATION, Seoul (KR)

(72) Inventors: Ki Chang Kim, Suwon-si (KR); Dong Chul Park, Anyang-si (KR)

(73) Assignees: HYUNDAI MOTOR COMPANY, Seoul (KR); KIA CORPORATION, Seoul (KR)

(21) Appl. No.: 18/227,677

(22) Filed: Jul. 28, 2023

(30) Foreign Application Priority Data
Dec. 16, 2022 (KR) 10-2022-0177523

(51) Int. Cl. B60W 60/00; A61B 5/00; A61B 5/16; A61B 5/369; B60W 50/14

(52) U.S. Cl. CPC B60W 60/005; A61B 5/165; A61B 5/369; A61B 5/4088; A61B 5/4803; A61B 5/746; B60W 50/14; B60W 2050/143; B60W 60/0016; B60W 2540/21; B60W 2540/22; B60W 2540/221

(57) ABSTRACT

A vehicle control apparatus and a method thereof are provided. The vehicle control apparatus includes a first detection device that detects biometric information of a passenger in a vehicle using a sensor and a processor electrically connected with the first detection device. The processor: determines whether to approve a predetermined autonomous driving level based on the biometric information; determines whether the passenger has dementia based on voice interaction in response to determining that the predetermined autonomous driving level is not approved; and determines whether to perform autonomous driving based on the result of determining whether the passenger has dementia.

### CROSS-REFERENCE TO RELATED APPLICATION

This application claims the benefit of priority to Korean Patent Application No. 10-2022-0177523, filed in the Korean Intellectual Property Office on Dec. 16, 2022, the entire contents of which are incorporated herein by reference.

### TECHNICAL FIELD

The present disclosure relates to a vehicle control apparatus and a method thereof.

### BACKGROUND

An autonomous vehicle refers to a vehicle capable of recognizing driving environments without manipulation by its driver to determine a risk and also of planning a driving route to drive itself. An autonomous driving technology loaded into such an autonomous vehicle is divided into six stages from Level 0 to Level 5 according to the guideline (J3016) presented by the Society of Automotive Engineers (SAE). From Level 0 to Level 2, a driver is included in an entity who controls the vehicle. Thus, when the driver, such as an elderly driver or a driver with underlying diseases (e.g., a dementia patient or the like), sets an autonomous driving level that is less than or equal to Level 2, it may be difficult to ensure the safety of a passenger who rides in the vehicle.

### SUMMARY

The present disclosure has been made to solve the above-mentioned problems occurring in the prior art while advantages achieved by the prior art are maintained intact.

An aspect of the present disclosure provides a vehicle control apparatus for determining whether a driver has dementia using biometric information and voice interaction and for controlling whether to perform autonomous driving depending on the determined result. Another aspect of the present disclosure provides a method thereof.

The technical problems to be solved by the present disclosure are not limited to the aforementioned problems. Any other technical problems not mentioned herein should be more clearly understood from the following description by those having ordinary skill art to which the present disclosure pertains.

According to an aspect of the present disclosure, a vehicle control apparatus may include a first detection device that detects biometric information of a passenger in a vehicle using a sensor and a processor electrically connected with the first detection device. The processor may determine whether to approve a predetermined autonomous driving level based on the biometric information, may determine whether the passenger has dementia based on voice interaction in response to determining that the predetermined autonomous driving level is not approved, and may determine whether to perform autonomous driving based on the result of determining whether the passenger has dementia.

The processor may turn off an autonomous driving function and may output a warning indicating that it is impossible to drive in response to determining that the passenger has dementia.

The processor may output a virtual sound based on a vehicle environment in response to determining that the predetermined autonomous driving level is approved.

The processor may tune the virtual sound in conjunction with the biometric information.

The processor may output a virtual sound based on an emotional state of the passenger in response to determining that the passenger does not have dementia.

The processor may compare an electroencephalogram (EEG) measurement value with a predetermined reference EEG value, when a peak is detected at a specified frequency of EEG. The processor may determine whether the predetermined autonomous driving level is less than or equal to a reference autonomous driving level, when a difference between the EEG measurement value and the reference EEG value is greater than or equal to a threshold. The processor may determine not to approve autonomous driving, when the predetermined autonomous driving level is less than or equal to the reference autonomous driving level.

The processor may obtain a physical parameter and an emotional parameter based on the voice interaction, when the difference between the EEG measurement value and the reference EEG value is not greater than or equal to the threshold. The processor may analyze a linguistic characteristic of the passenger based on the physical parameter and the emotional parameter. The processor may compare the linguistic characteristic of the passenger with a linguistic characteristic of a dementia patient. The processor may determine that the predetermined autonomous driving level is less than or equal to the reference autonomous driving level, when a similarity between the linguistic characteristic of the passenger and the linguistic characteristic of the dementia patient is greater than or equal to a predetermined reference value. The processor may determine not to approve autonomous driving in response to determining that the predetermined autonomous driving level is less than or equal to the reference autonomous driving level.

The processor may detect an arrhythmia pattern based on a heart rate signal, when the peak is not detected at the specific frequency of the EEG. The processor may detect breathing instability based on a breathing rate and a breathing sound, may compare the EEG measurement value with the reference EEG value, when the arrhythmia pattern and the breathing instability are detected. The processor may determine whether to approve the reference autonomous driving level based on the result of comparing the EEG measurement value with the reference EEG value.

The biometric information may include at least one of an EEG, heart rate, blood pressure, breathing sound, body temperature, or a combination thereof.

The first detection device may include at least one of a non-contact EEG sensor, a heart rate sensor, a body temperature sensor, a blood pressure sensor, a microphone, or a combination thereof.

According to another aspect of the present disclosure, a vehicle control method may include: detecting biometric information of a passenger in a vehicle using a sensor; determining whether to approve a predetermined autonomous driving level based on the biometric information; determining whether the passenger has dementia based on voice interaction in response to determining that the predetermined autonomous driving level is not approved; and determining whether to perform autonomous driving based on the result of determining whether the passenger has dementia.

Determining whether to perform autonomous driving may include turning off an autonomous driving function in response to determining that the passenger has dementia and outputting a warning indicating that it is impossible to drive.

The vehicle control method may further include outputting a virtual sound based on vehicle environment information in response to determining that the predetermined autonomous driving level is approved.

Outputting the virtual sound may include tuning the virtual sound in conjunction with the biometric information.

Determining whether to perform autonomous driving may include outputting a virtual sound based on an emotional state of the passenger in response to determining that the passenger does not have dementia.

Determining whether the passenger has dementia may include: comparing an EEG measurement value with a predetermined reference EEG value, when a peak is detected at a specified frequency of EEG; determining whether the predetermined autonomous driving level is less than or equal to a reference autonomous driving level, when a difference between the EEG measurement value and the reference EEG value is greater than or equal to a threshold; and determining not to approve autonomous driving in response to determining that autonomous driving level is less than or equal to the reference autonomous driving level.

Determining whether the passenger has dementia may include: obtaining a physical parameter and an emotional parameter based on the voice interaction, when the difference between the EEG measurement value and the reference EEG value is not greater than or equal to the threshold; analyzing a linguistic characteristic of the based on the physical parameter and the emotional parameter; comparing the linguistic characteristic of the passenger with a linguistic characteristic of a dementia patient; and determining not to approve the reference autonomous driving level or less, when a similarity between the linguistic characteristic of the passenger and the linguistic characteristic of the patient with dementia is greater than or equal to a predetermined reference value.

Determining whether the passenger has dementia may include: detecting an arrhythmia pattern based on a heart rate signal, when the peak is not detected at the specific frequency of the EEG; detecting breathing instability based on a breathing rate and a breathing sound; comparing the EEG measurement value with the reference EEG value, when the arrhythmia pattern and the breathing instability are detected; and determining whether to approve the reference autonomous driving level based on the result of comparing the EEG measurement value with the reference EEG value.

### BRIEF DESCRIPTION OF THE DRAWINGS

The above and other objects, features, and advantages of the present disclosure should be more apparent from the following detailed description taken in conjunction with the accompanying drawings:

FIG. 1 is a block diagram illustrating a configuration of a vehicle control apparatus according to embodiments of the present disclosure;

FIG. 2 is a drawing for describing a process of classifying passenger emotion according to embodiments of the present disclosure;

FIG. 3 is a flowchart illustrating a vehicle control method according to embodiments of the present disclosure;

FIG. 4 is a flowchart illustrating a process of determining dementia according to embodiments of the present disclosure; and

FIG. 5 is a flowchart illustrating a method for diagnosing dementia according to embodiments of the present disclosure.

### DETAILED DESCRIPTION

Hereinafter, some embodiments of the present disclosure are described in detail with reference to the drawings. In the drawings, the same reference numerals are used throughout to designate the same or equivalent elements. In addition, a detailed description of well-known features or functions has been ruled out in order not to unnecessarily obscure the gist of the present disclosure. When a component, device, element, or the like, of the present disclosure, is described as having a purpose or performing an operation, function, or the like, the component, device, or element should be considered herein as being “configured to” meet that purpose or to perform that operation or function.

In describing the components of embodiments according to the present disclosure, terms such as first, second, “A”, “B”, (a), (b), and the like may be used. These terms are only used to distinguish one element from another element but do not limit the corresponding elements irrespective of the order or priority of the corresponding elements. Furthermore, unless otherwise defined, all terms including technical and scientific terms used herein are to be interpreted as is customary in the art to which the present disclosure belongs. Such terms as those defined in a generally used dictionary are to be interpreted as having meanings consistent with the contextual meanings in the relevant field of art and are not to be interpreted as having ideal or excessively formal meanings unless clearly defined as having such in the present application.

Embodiments of the present disclosure relate to a technology of determining whether a passenger (e.g., a driver, another passenger, or the like) who rides in an autonomous vehicle has dementia based on biometric information of the passenger and voice interaction and of controlling whether to perform autonomous driving based on the determined result. The level of automation of the autonomous vehicle, i.e., an autonomous driving level may be divided into six stages from Level 0 to Level 5 according to the standard presented by the Society of Automotive Engineers (SAE). Level 0 is defined as the no automation stage (stage 1). Level 1 is defined as the driver assistance stage (stage 2). Level 2 is defined as the partial automation stage (stage 3). Level 3 is defined as the partial automation stage (stage 4). Level 4 is defined as the high automation stage (stage 5). Level 5 is defined as the full automation stage (stage 6).

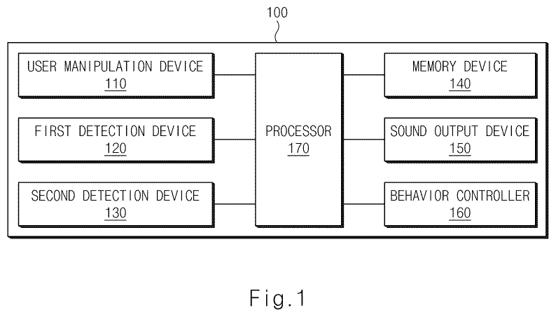

FIG. 1 is a block diagram illustrating a configuration of a vehicle control apparatus according to embodiments of the present disclosure.

A vehicle control apparatus 100 may be loaded into a vehicle capable of performing autonomous driving. The vehicle control apparatus 100 may include a user manipulation device 110, a first detection device 120, a second detection device 130, a memory device 140, a sound output device 150, a behavior controller 160, and a processor 170.

The user manipulation device 110 may generate data according to user manipulation. The user manipulation device 110 may generate a control command according to a user input. For example, when a driver manipulates a steering input device (e.g., a steering wheel), an acceleration input device (e.g., an accelerator pedal), a braking input device (e.g., a brake pedal), and/or the like, the user manipulation device 110 may generate a steering command, an acceleration command, a braking command, and/or the like according to the manipulation.

Furthermore, the user manipulation device 110 may include devices (e.g., a button, a touch pad, a touch screen, or the like) for turning on/off an emotional care solution, setting an autonomous driving level, starting a vehicle, and manipulating vehicle functions such as navigation, a seat heating wire, a turn signal, a chiller/heater, wipers, and the like.

The first detection device 120 may detect biometric information of a passenger using a sensor. The biometric information may include at least one of an electroencephalogram (EEG), heart rate, blood pressure, breathing sound, body temperature, or a combination thereof. The first detection device 120 may obtain biometric information using at least one of an EEG sensor, a heart rate sensor, a blood pressure sensor, a body temperature sensor (or a temperature sensor), a respiration sensor, or a combination thereof.

The EEG sensor may be a non-contact sensing device using a radar, which may be mounted on a vehicle seat. The EEG sensor may generate a signal and may analyze information reflected from at least a portion of the human body. The EEG sensor may include at least two or more electric field sensors, which may perform non-contact ECG and EEG measurement when detecting an electric field by a heartbeat. As an electric field and a magnetic field are generated around a location in which current flows, the electric field sensor may detect an electric field proportional to a biocurrent.

The heart rate sensor may measure a heart rate on a wrist or an ear of a passenger. The heart rate may be used to determine the stress of the driver or diagnose emotion of the driver. The heart rate may vary with gender, age, an exercise state, an emotional state, and/or a surrounding environment and may refer to a maximum heart rate.

The respiration sensor may measure a breathing rate and/or a breathing sound using a microphone. The breathing rate may be used to determine a health state of the driver, and the breathing sound may be used to determine the stress of the driver or diagnose dementia of the driver. The respiration sensor may determine a degree of loudness and roughness of a breathing sound by analyzing a pattern of a breathing sound source.

The second detection device 130 may detect vehicle environment information (or driving environment information), passenger information, and/or the like by means of sensors and/or electronic control units (ECUs) loaded into the vehicle. The vehicle environment information may include a driver steering angle (or a steering wheel steering angle), a tire steering angle (or a tie rod angle), vehicle speed, motor revolutions per minute (RPM), motor torque, an accelerator pedal opening amount, and/or the like. An accelerator position sensor (APS), a steering angle sensor, a microphone, an image sensor, a distance sensor, a wheel speed sensor, an advanced driver assistance system (ADAS) sensor, a 3-axis accelerometer, an inertial measurement unit (IMU), and/or the like may be used as the sensors. A motor control unit (MCU), a vehicle control unit (VCU), and/or the like may be used as ECUs.

The memory device 140 may store a sound design algorithm, a volume setting algorithm, an emotion classifier (or an emotion analysis algorithm), a dementia diagnosis (or determination) algorithm, a previously learned model (or a big data-based emotion classification model), a language model, a three-dimensional (3D) sound analysis model, and/or the like. The emotion classifier is a model designed based on a conversational memory network (CMN). The emotion classifier may convert 360 emotion proposals into concrete attributes based on a sound. The memory device 140 may store an emotional content, a virtual sound, and/or the like.

The memory device 140 may be a non-transitory memory that stores instructions executed by the processor 170. The memory device 140 may include at least one of storage media such as a random access memory (RAM), a static RAM (SRAM), a read only memory (ROM), read only memory a programmable ROM (PROM), an electrically erasable and programmable ROM (EEPROM), an erasable and programmable ROM (EPROM), a hard disk drive (HDD), a solid state disk (SSD), an embedded multimedia card (eMMC), universal flash storage (UFS), or web storage.

The sound output device 150 may play and output a virtual sound through speakers mounted on the inside and/or outside of the vehicle. The sound output device 150 may play and output a sound source which is previously stored or is streamed in real time. The sound output device 150 may include an amplifier, a sound playback device, and the like. The sound playback device may adjust and play volume, timbre (or sound quality), a sound image, and the like of the sound under an instruction of the processor 170. The sound playback device may include a digital signal processor (DSP), microprocessors, and/or the like. The amplifier may amplify an electrical signal of the sound played from the sound playback device.

The behavior controller 160 may control a behavior of the vehicle, for example, acceleration, steering, braking, and the like under an instruction of the processor 170. The behavior controller 160 may include a driving controller, a braking controller, a steering controller, a shift controller, and the like. The driving controller, the braking controller, the steering controller, and the shift controller may be implemented as one electronic control unit (ECU) or may be implemented as separate ECUs, respectively. The ECU may include a memory for storing software programmed to perform a predetermined function (or operation), a processor for executing the software stored in the memory, and the like. The driving controller may control a power source (e.g., an engine, a driving motor, and the like) of the vehicle. The driving controller may control power (e.g., power torque) of a power source depending on accelerator pedal position information or a driving speed required from the processor 170. The driving controller may include an engine management system (EMS), a motor control unit (MCU), and/or the like. The braking controller may be to control deceleration (or braking) of the vehicle, which may control a braking force of the vehicle based on a brake pedal position or a required braking force of the processor 170. The braking controller may include electronic stability control (ESC) or the like. The steering controller may be to control steering of the vehicle, which may include motor drive power steering (MDPS) or the like. The shift controller may be to control a transmission of the vehicle, which may adjust a shift ratio depending on a gear position and/or a gear state range. The shift controller may be implemented as a transmission control unit (TCU) or the like.

The processor 170 may be electrically connected with the respective components 110 to 160. The processor 170 may include at least one of processing devices such as an application specific integrated circuit (ASIC), a digital signal processor (DSP), programmable logic devices (PLD), field programmable gate arrays (FPGAs), a central processing unit (CPU), microcontrollers, or microprocessors.

The processor 170 may receive driver manipulation information (or a user input or a passenger input) from the user manipulation device 110. The driver manipulation information may include ignition on or off information, destination setting information, and the like. Furthermore, the driver manipulation information may include autonomous driving level setting information.

The processor 170 may determine dementia of a passenger who rides in the vehicle. The processor 170 may perform primary dementia determination based on biometric information and may perform secondary dementia determination based on voice interaction.

The processor 170 may obtain the biometric information of the passenger who rides in the vehicle by means of the first detection device 120. The processor 170 may use an EEG signal measured by the non-contact EEG measurement device as main data and may use a heart rate measured by the heart rate measurement device and a change in body temperature measured by the body temperature measurement device as sub-data to determine emotion and dementia of the passenger.

As an example, the processor 170 may determine emotion (or an emotional state) of a driver based on the EEG sensed in a non-contact manner, the heart rate, and the change in body temperature. Furthermore, the processor 170 may design and play an ultra-realistic sound in conjunction with a low speed, acceleration, and/or a traffic congestion situation by establishing three types of emotion modeling.

The processor 170 may analyze the EEG signal measured by the non-contact EEG measurement device and may determine an emotional state of the passenger. The processor 170 may analyze the heart rate signal measured by the heart rate measurement device and may detect arrhythmia. Furthermore, the processor 170 may analyze the breathing sound measured by the microphone and may detect a breathing pattern.

The processor 170 may analyze the biometric information detected by the first detection device 120 and may determine whether an autonomous driving level predetermined by the passenger is an approvable level based on the analyzed result. The processor 170 may determine whether the passenger has dementia based on the biometric information. When the passenger is suspected of having dementia, the processor 170 may identify whether the predetermined autonomous driving level is less than or equal to a reference autonomous driving level (e.g., Level 2). When the predetermined autonomous driving level is less than or equal to the reference autonomous driving level (e.g., Level 2), the processor 170 may turn off an autonomous driving function (or mode) and may output a warning (e.g., a warning message and/or a warning sound) indicating that it is impossible to drive.

When an EEG peak is detected at a predetermined specific frequency as a result of measuring the EEG, the processor 170 may analyze the EEG in detail. The processor 170 may determine whether a difference between the measured EEG measurement value of the passenger (or the driver) and a reference EEG value is greater than or equal to a threshold (e.g., 20%). Herein, the reference EEG value may be determined as an average EEG value of healthy persons in advance. When it is determined that the difference between the EEG measurement value of the passenger and the reference EEG value is greater than or equal to the threshold, the processor 170 may determine not to approve the reference autonomous driving level or less. When it is determined that the difference between the EEG measurement value of the passenger and the reference EEG value is not greater than or equal to the threshold, the processor 170 may determine to approve the reference autonomous driving level or less. The reference autonomous driving level may be determined in advance by a system designer. When it is determined not to approve the reference autonomous driving level or less, the processor 170 may determine whether the autonomous driving level set by the driver is less than or equal to the reference autonomous driving level. When the autonomous driving level set by the driver is less than or equal to the reference autonomous driving level, the processor 170 may determine not to approve the autonomous driving level set by the driver. For example, when the reference autonomous driving level is Level 2, when driving is not approved below the reference autonomous driving level, and when the autonomous driving level set by the driver is Level 2, the processor 170 may determine disapproval such that the driver is unable to drive in the autonomous driving level.

The processor 170 may determine whether the passenger has dementia based on voice interaction. The processor 170 may determine whether the driver is in a dementia determination level using a dementia diagnosis algorithm (or a health care algorithm) based on voice interaction. The processor 170 may diagnose whether the driver is in the dementia determination level by calculating sound (or audio)-based text using deep learning. The emotion analysis algorithm may encode an utterance corresponding to each speaker to derive a result by means of calculation with a target sentence. The 360 emotion proposals may be converted into concrete attributes by means of the sound-based emotion classifier. The concrete attributes may be associated with a keyword, such as classic music, a game sound, a racing car, or a family voice, to use hearing experience in future cars. For example, when the driver utters “when accelerating, I can feel the sound of the engine, it is fun and tastes like driving.”, the emotion classifier may output “a sound like a racing game” as a related keyword.

The processor 170 may receive an audio signal uttered by the passenger in a traffic accident state, a traffic congestion state, and/or a driving state (e.g., a constant speed or acceleration) and may convert the received audio signal into text. The processor 170 may analyze the converted text and may determine emotion of a passenger (i.e., a speaker). The processor 170 may classify an emotional state of the passenger using the emotion classifier, which is the CMN-based emotion model.

As an example, when only the driver rides in the vehicle, the processor 170 may output a voice interaction-based question and may receive a response from the driver to the interaction-based question. The processor 170 may classify an emotional state of the driver depending on the response of the driver to the voice interaction-based question.

As another example, when the driver and at least one passenger ride in the vehicle, the processor 170 may receive a conversion between the driver and the at least one passenger as a sound source. The processor 170 may analyze conversion contents between the driver and the at least one passenger and may classify an emotional state of the driver. The processor 170 may also classify an emotional state of the at least one passenger.

The processor 170 may determine a driving environment (or a driving situation) based on pieces of information obtained using a camera, light detection and ranging (LiDAR), an ultrasonic sensor, and/or the like. Herein, the driving environment may be classified as smooth traffic, traffic congestion, an accident risk, and/or the like. The processor 170 may play music content with regard to the driving environment.

As an example, in the smooth traffic situation, the processor 170 may output a voice interaction-based artificial intelligence (AI) question, for example, “It's a warm spring day. Please tell me if you have any memories that come to mind right now.”, through a speaker. When receiving a response (or an answer) to it, “Remind me of memories with my girlfriend at the cherry blossom festival”, the processor 170 may play a healing sound.

As another example, in the traffic generation situation, the processor 170 may output a voice interaction-based AI question, “Traffic is very congested. Please express in words how frustrated you are right now.”. When receiving an answer to it, “My heart is stuffy and I'm under a lot of stress.”, the processor 170 may play a classical sound.

As another example, in the accident risk situation, the processor 170 may output a voice interaction-based AI question, “Wow, that almost was a big deal. Please tell me about the situation and how surprised you were.”. When receiving a response, “Oh, I almost died, I escaped a car accident.”, the processor 170 may play a meditation sound.

As another example, when there is no response of the passenger to the voice interaction-based AI question or when stuttering of the passenger is detected, because the passenger is suspected of having dementia, the processor 170 may perform additional verification.

As another example, the processor 170 may measure EEG and may analyze a pattern of the measured EEG signal. Furthermore, the processor 170 may measure a heart rate signal and may analyze a heart rate pattern. The processor 170 may determine an emotional state matched with the EEG pattern and the heart rate pattern using the emotion diagnosis algorithm. The processor 170 may determine whether the passenger is suspected of having dementia based on the emotional state matched with the EEG pattern and the heart rate pattern. When the emotional state according to the EEG pattern is “depression” and when the emotional state according to the heart rate pattern is “sadness and depression”, or when the emotional state according to the EEG pattern is “anger” and when the emotional state according to the heart rate pattern is “anger”, the processor 170 may determine that the passenger is suspected of having dementia. When the passenger is suspected of having dementia, the processor 170 may perform a verification procedure based on voice interaction.

The processor 170 may analyze a facial expression of the passenger by means of a camera (or an interior camera) mounted to capture the interior of the vehicle and may recognize an emotional state of the passenger. The processor 170 may identify whether there is a history of suspected dementia on a medical history database (DB) of the passenger. When there is the history of suspected dementia in the medical history DB of the passenger and when the result of determining the emotional state according to the EEG analysis is “depression” and the result of determining the emotional state based on the voice interaction is “sadness”, the processor 170 may determine that the passenger has dementia.

Furthermore, the processor 170 may recognize a driving environment by means of a camera (or an exterior camera) mounted to capture the exterior of the vehicle. In a situation where the driving environment is a traffic accident and/or traffic congestion, the processor 170 may fail to determine dementia by using only voice interaction data. In a situation where the driving environment is not the traffic accident and/or the traffic congestion, when the emotional state according to the EEG analysis is determined as “anger” and when the emotional state based on the voice interaction is “fear” or “anger”, the processor 170 may determine that the passenger is on dementia boundary.

The processor 170 may tune a sound in conjunction with biometric information by means of artificial neural network (ANN)-based learning. The ANN may create an important value by analyzing psychosocial consequences and may use it to establish an emotional model for development of auditory experience for future cars.

The processor 170 may derive a physical parameter (or factor) from the biometric information. The physical parameter may include a frequency per second, a magnitude of a signal, a height of the a pattern of the signal, or signal, regularity/irregularity. The processor 170 may derive an emotional parameter from voice information. The emotional parameter may include whether the sound is comfortable, whether the sound is appropriate, whether the sense of speed is felt, harmony with ambient noise, or a change in user mood.

The processor 170 may analyze the physical and emotional parameters and may recommend a personalized sound based on the analyzed result. The personalized sound may include a meditation sound for determining and relieve stress due to traffic congestion while the vehicle is traveling, a healing sound which interworks with a surrounding environment while the vehicle is traveling at a constant speed, a fun sound for stimulating driving sensibility while the vehicle is accelerating, or the like.

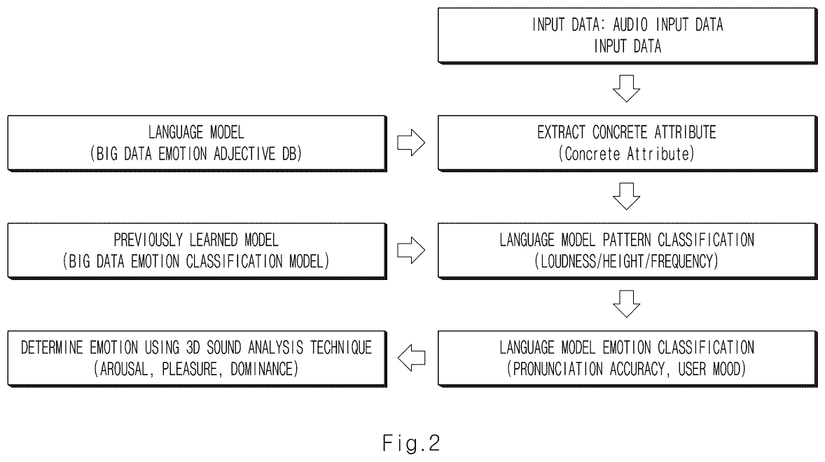

FIG. 2 is a drawing for describing a process of classifying passenger emotion according to embodiments of the present disclosure.

An emotion classifier may classify (or analyze) emotion of a passenger (e.g., a driver) based on a CMN.

The emotion classifier may receive audio data (or an audio signal) as input data. The emotion classifier may generate a single or conversational utterance of the driver as a single sound source (or single audio data). The emotion classifier may convert the audio data (e.g., a waveform audio or WAV file) into text data.

The emotion classifier may extract a concrete attribute (or feature) from the audio data. The concrete attribute may be used to analyze volume and timbre of the audio data.

The emotion classifier may perform language model pattern classification. The emotion classifier may determine (or analyze) a physical parameter of the audio data, for example, loudness, height, a frequency, and the like. The emotion classifier may determine the physical parameter of the audio data using a big data emotion classification model which is a previously learned model.

The emotion classifier may perform language model emotion classification. The emotion classifier may determine an emotional parameter of the audio data, for example, pronunciation accuracy, a user mood, or the like.

The emotion classifier may determine an emotional state based on the result of performing the language model pattern classification and the result of performing the language model emotion classification. The emotion classifier may classify an emotional state using a 3D sound analysis technique.

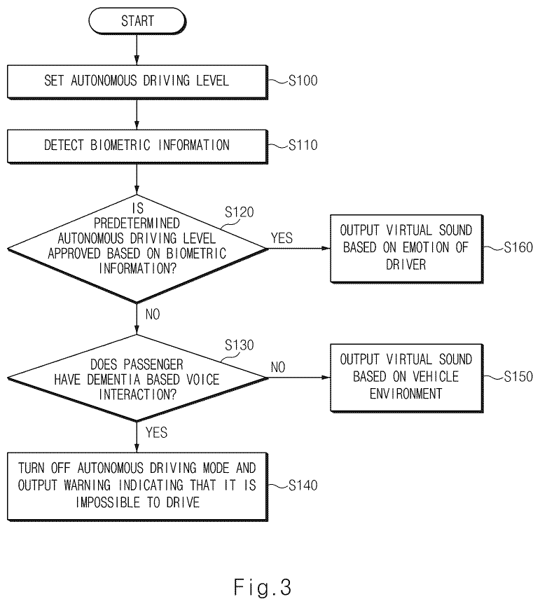

FIG. 3 is a flowchart illustrating a vehicle control method according to embodiments of the present disclosure.

In S100, a processor 170 of FIG. 1 may set an autonomous driving level depending on a user input received from a user manipulation device 110 of FIG. 1.

In S110, the processor 170 may detect biometric information of a passenger who rides in a vehicle. The processor 170 may detect biometric information for a biometric information analysis at a time point when a stress situation is detected by an external camera in a state where an emotional care solution mode is set to ON or when a change in biometric signal is detected above a predetermined rate (20%).

In S120, the processor 170 may determine whether to approve a predetermined autonomous driving level based on the biometric information. The processor 170 may analyze EEG, a heart rate, breath, and/or the like and may determine whether to approve the predetermined autonomous driving level based on the analyzed result. Herein, the predetermined autonomous driving level refers to an autonomous driving level set by a user input, i.e., an input of a driver in S100.

When it is determined not to approve the predetermined autonomous driving level (NO in S120), in S130, the processor 170 may determine whether the passenger has dementia based on voice interaction. The processor 170 may convert a voice uttered by the passenger into text and may analyze the converted text to determine whether the passenger has dementia. As an example, when only the driver rides in the vehicle, the processor 170 may output an AI question based on voice interaction and may receive and analyze a response of the driver to the AI question, thus determining whether the driver has dementia. For example, when the driver is unable to answer the AI question or stutters, the processor 170 may determine that the driver has dementia. As another example, when a passenger other than the driver rides in the vehicle, the processor 170 may analyze a conversion between the driver and the passenger and may determine whether the driver has dementia.

When it is determined that the passenger has dementia (YES in S130), in S140, the processor 170 may turn off an autonomous driving mode (or function) and may output a warning indicating that it is impossible to drive. The processor 170 may output a warning message indicating that it is impossible to drive on a display or may output a warning sound through a speaker. Furthermore, the processor 170 may output the warning message indicating that it is impossible to drive on the display and may output the warning sound through the speaker.

When it is determined that the passenger does not have dementia (NO in S130), in S150, the processor 170 may output a virtual sound based on emotion of the driver. The processor 170 may apply (or assign) an emotional content concept (or meaning) to the virtual sound based on the result of analyzing a physical parameter of biometric information and the result of analyzing an emotional parameter of voice information (or information based on voice interaction).

When it is determined to approve the autonomous driving level (YES in S120), in S160, the processor 170 may output a virtual sound based on a vehicle environment (or a driving environment). The processor 170 may tune the virtual sound in conjunction with the biometric information.

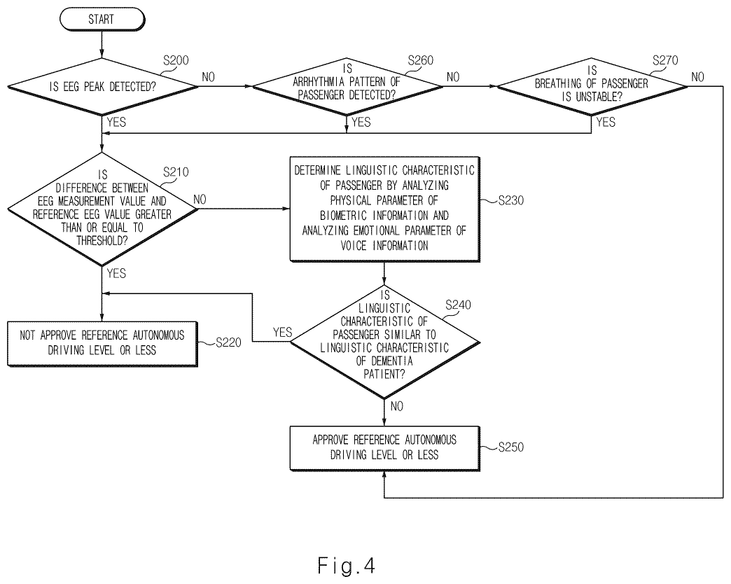

FIG. 4 is a flowchart illustrating a process of determining dementia according to embodiments of the present disclosure.

In S200, a processor 170 of FIG. 1 may detect an EEG peak at a predetermined specific frequency of an EEG signal. The processor 170 may analyze EEG received from an EEG sensor and may extract the EEG peak.

When the EEG peak is detected (YES in S200), in S210, the processor 170 may determine whether a difference between EEG measurement value and a reference EEG value is greater than or equal to a threshold (e.g., 20%). The reference EEG value may be determined as an average EEG value of healthy persons.

When it is determined that the difference between the EEG measurement value and the reference EEG value is greater than or equal to the threshold (YES in S210), in S220, the processor 170 may determine not to approve a reference autonomous driving level or less. The reference autonomous driving level may be set in advance by a system designer. When the autonomous driving level set by a driver is less than or equal to the reference autonomous driving level, the processor 170 may determine not to approve autonomous driving of a vehicle.

When it is determined that the difference between the EEG measurement value and the reference EEG value is not greater than or equal to the threshold (NO in S210), in S230, the processor 170 may determine a linguistic characteristic of a passenger by analyzing a physical parameter of biometric information and analyzing an emotional parameter of voice information.

In S240, the processor 170 may determine whether the linguistic characteristic of the passenger is similar to a linguistic characteristic of a dementia patient. When a similarity between the linguistic characteristic of the passenger and the linguistic characteristic of the dementia patient is greater than or equal to a predetermined reference value, the processor 170 may determine that the linguistic characteristic of the passenger is similar to the linguistic characteristic of the dementia patient (YES in S240).

When it is determined that the linguistic characteristic of the passenger is similar to the linguistic characteristic of the dementia patient, in S220, the processor 170 may determine not to approve the reference autonomous driving level or less.

When it is determined that the linguistic characteristic of the passenger is not similar to the linguistic characteristic of the dementia patient (NO in S240), in S250, the processor 170 may determine to approve the reference autonomous driving level or less.

When the EEG peak is not detected (NO in S200), in S260, the processor 170 may determine whether an arrhythmia pattern of the passenger is detected. The processor 170 may analyze a heart rate signal of the passenger and may determine whether the passenger has arrhythmia.

When the arrhythmia pattern is not detected (NO in S260), in S270, the processor 170 may determine whether the breathing of the passenger is unstable. The processor 170 may analyze a breathing rate, a breathing sound, and the like of the passenger and may determine whether the breathing of the passenger is unstable.

When the arrhythmia pattern is detected (YES in S260) or when the unstable breathing is detected (YES in S270), the processor 170 may perform the operation from S210. When the unstable breathing is not detected (NO in S270), in S250, the processor 170 may determine to approve the reference autonomous driving level or less.

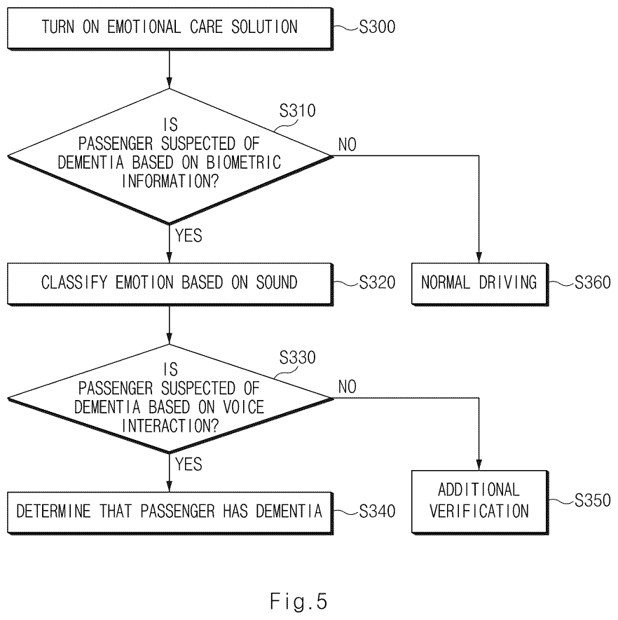

FIG. 5 is a flowchart illustrating a method for diagnosing dementia according to embodiments of the present disclosure.

In S300, a processor 170 of FIG. 1 may turn on an emotional care solution depending on a user input received from a user manipulation device 110 of FIG. 1.

In S310, the processor 170 may determine whether a passenger is suspected of having dementia based on biometric information. The processor 170 may identify whether there is a history of suspected dementia in a medical history DB of the passenger with reference to the medical history DB of the passenger. When there is the history of suspected dementia, the processor 170 may analyze biometric information of the passenger, which is measured by a sensor, and may determine whether the passenger has dementia.

When it is determined that the passenger is suspected of having dementia (YES in S310), in S320, the processor 170 may classify an emotional state of the passenger based on a sound. The processor 170 may analyze a voice of the passenger using an emotion diagnosis algorithm (or an emotion classifier) and may classify an emotional state of the passenger. The emotion diagnosis algorithm may learn loudness (or volume), accuracy of a word (or timbre), and a frequency characteristic when the word is uttered based on a usual conversation of the passenger.

In S330, the processor 170 may determine whether the passenger is suspected of having dementia based on voice interaction. The processor 170 may output a question based on voice interaction to the passenger and may receive and analyze a response of the passenger. The processor 170 may determine whether the passenger is suspected of having dementia based on the analyzed result.

When it is determined again that the passenger is suspected of having dementia (YES in S330), in S340, the processor 170 may determine that the passenger has dementia. The processor 170 may turn off an autonomous driving function and may output a warning indicating that it is impossible to drive. Furthermore, the processor 170 may provide a 911 center or the like with information indicating that the passenger has dementia using a communication function (e.g., a transceiver).

When it is determined again that the passenger is not suspected of having dementia (NO in S330), in S350, the processor 170 may perform additional verification. The processor 170 may determine whether the passenger has dementia by means of emotion interworking based on biometric information and voice interaction.

As an example, when EEG and a heart rate of the driver have a difference above a threshold (e.g., 20%) compared to an average value of healthy persons, the processor 170 may analyze an EEG pattern and a heart rate pattern and may determine an emotional state of the passenger. When the emotional state according to the EEG pattern is “depression” and when the emotional state according to the heart rate pattern is “sadness and depression”, or when the emotional state according to the EEG pattern is “anger” and when the emotional state according to the heart rate pattern is “anger”, the processor 170 may determine that the passenger has a history of suspected dementia with reference to the medical history DB of the passenger. Furthermore, when a linguistic characteristic of the passenger is similar to a linguistic characteristic of a dementia patient as a result of the analysis based on the voice interaction, the processor 170 may analyze a voice pattern of the passenger and may classify an emotional state of the passenger. When the classified emotional state is sadness or fear and anger, the processor 170 may identify a driving environment by means of an external camera. When the passenger has the history of suspected dementia, when it is determined that the passenger is a “depression” state as a result of analyzing the EEG pattern, and when it is determined that the passenger is in a “sadness” state as a result of analyzing the voice pattern, the processor 170 may determine that the passenger has dementia. When the driving environment is not a traffic accident situation and/or a traffic congestion situation, when it is determined that the passenger is in an “anger” state as a result of analyzing the EEG pattern, and when it is determined that the passenger is in a “fear and anger” state as a result of analyzing the voice pattern, the processor 170 may determine that the passenger is on a dementia boundary.

As another example, when EEG and a heart rate of the driver have a difference above the threshold (e.g., 20%) compared to the average value of healthy persons, the processor 170 may analyze an EEG pattern and a heart rate pattern with reference to the medical history DB of the passenger. In the state where the passenger has a history of suspected dementia, when the passenger is in a “depression” state as a result of analyzing the EEG pattern, is in a “sadness and depression” state as a result of analyzing the heart rate pattern, and is in a “sadness” state as a result of analyzing the voice pattern, the processor 170 may determine that the passenger has dementia. When the passenger has dementia, the processor 170 may determine not to approve the reference autonomous driving level or less and may deliver information to a 911 center using a communication function. Furthermore, when a linguistic characteristic of the passenger is similar to a linguistic characteristic of a dementia patient as a result of the analysis based on the voice interaction, the processor 170 may identify a driving environment by means of an external camera. In a situation where the driving environment is not a traffic accident and/or traffic congestion, when the passenger is in an “anger” state as a result of analyzing the EEG pattern, is in a “anger” state as a result of analyzing the heart rate pattern, and is in a “fear and anger” state as a result of analyzing the voice pattern, the processor 170 may determine that the passenger is on a dementia boundary. When it is determined that the passenger is on the dementia boundary, the processor 170 may play and output a relax sound to the passenger.

When it is determined that the passenger is not suspected of having dementia (NO in S310), in S360, the processor 170 may perform normal driving.

The above-mentioned embodiments disclose that the dementia possibility determination based on the biometric signal and the dementia possibility determination based on the voice interaction are performed in series but may be implemented to perform them in parallel.

Furthermore, the above-mentioned embodiments may analyze volume, timbre, and a frequency of an audio signal measured by a sensor when a target sentence is uttered using a biometric signal and voice analysis DB of the passenger based on deep learning and may determine an emotional state of the passenger and whether the passenger has dementia.

Embodiments of the present disclosure may determine whether the driver has dementia using biometric information and voice interaction and may control whether to perform autonomous driving depending on the determined result, thus preventing a critical situation where the driver who is suspected of having dementia drives the vehicle.

Furthermore, embodiments of the present disclosure may provide a healthcare solution for guiding a passenger who rides in an autonomous vehicle to improve a sensation of immersion with regard to a health state of the passenger.

Furthermore, embodiments of the present disclosure may provide a notification function and a guide based on determination of whether it is suitable to drive in conjunction with a user terminal.

Furthermore, embodiments of the present disclosure may provide a scenario with regard to cardiac arrest or an emergency situation of an elderly driver and a driver with underlying diseases.

Hereinabove, although the present disclosure has been described with reference to embodiments and the accompanying drawings, the present disclosure is not limited thereto. The embodiments may be variously modified and altered by those having ordinary skill in the art to which the present disclosure pertains without departing from the spirit and scope of the present disclosure claimed in the following claims. Therefore, embodiments oft present disclosure are not intended to limit the technical spirit of the present disclosure but are provided only for illustrative purposes. The scope of the present disclosure should be construed on the basis of the accompanying claims, and all the technical ideas within the scope equivalent to the claims should be included in the scope of the present disclosure.

What is claimed is:

1. A vehicle control apparatus, comprising:
a first detection device configured to detect biometric information of a passenger in a vehicle using a sensor; and
a processor electrically connected with the first detection device,
wherein the processor is configured to
determine whether to approve a predetermined autonomous driving level based on the biometric information,
determine whether the passenger has dementia based on voice interaction in response to determining that the predetermined autonomous driving level is not approved, and
determine whether to perform autonomous driving based on the result of determining whether the passenger has dementia.

2. The vehicle control apparatus of claim 1, wherein the processor is configured to turn off an autonomous driving function and output a warning indicating that it is impossible to drive in response to determining that the passenger has dementia.

3. The vehicle control apparatus of claim 1, wherein the processor is configured to output a virtual sound based on a vehicle environment in response to determining that the predetermined autonomous driving level is approved.

4. The vehicle control apparatus of claim 3, wherein the processor is configured to tune virtual sound in conjunction with the biometric information.

5. The vehicle control apparatus of claim 1, wherein the processor is configured to output a virtual sound based on an emotional state of the passenger in response to determining that the passenger does not have dementia.

6. The vehicle control apparatus of claim 1, wherein the processor is configured to:
compare an electroencephalogram (EEG) measurement value with a predetermined reference EEG value, when a peak is detected at a specified frequency of EEG;
determine whether the predetermined autonomous driving level is less than or equal to a reference autonomous driving level, when a difference between the EEG measurement value and the reference EEG value is greater than or equal to a threshold; and
determine not to approve autonomous driving, when the predetermined autonomous driving level is less than or equal to the reference autonomous driving level.

7. The vehicle control apparatus of claim 6, wherein the processor is configured to:
obtain a physical parameter and an emotional parameter based on the voice interaction, when the difference between the EEG measurement value and the reference EEG value is not greater than or equal to the threshold;
analyze a linguistic characteristic of the passenger based on the physical parameter and the emotional parameter;
compare the linguistic characteristic of the passenger with a linguistic characteristic of a dementia patient;
determine that the predetermined autonomous driving level is less than or equal to the reference autonomous driving level, when a similarity between the linguistic characteristic of the passenger and the linguistic characteristic of the dementia patient is greater than or equal to a predetermined reference value; and
determine not to approve autonomous driving in response to determining that the predetermined autonomous driving level is less than or equal to the reference autonomous driving level.

8. The vehicle control apparatus of claim 6, wherein the processor is configured to:
detect an arrhythmia pattern based on a heart rate signal, when the peak is not detected at the specific frequency of the EEG;
detect breathing instability based on a breathing rate and a breathing sound;
compare the EEG measurement value with the reference EEG pattern and the breathing value, when the arrhythmia instability are detected; and
determine whether to approve the reference autonomous driving level based on the result of comparing the EEG measurement value with the reference EEG value.

9. The vehicle control apparatus of claim 1, wherein the biometric information includes at least one of an EEG, heart rate, blood pressure, breathing sound, body temperature, or a combination thereof.

10. The vehicle control apparatus of claim 1, wherein the first detection device includes at least one of a non-contact EEG sensor, a heart rate sensor, a body temperature sensor, a blood pressure sensor, a microphone, or a combination thereof.

11. A vehicle control method, comprising:
detecting biometric information of a passenger in a vehicle using a sensor;
determining whether to approve a predetermined autonomous driving level based on the biometric information;
determining whether the passenger has dementia based on voice interaction in response to determining that the predetermined autonomous driving level is not approved; and
determining whether to perform autonomous driving based on the result of determining whether the passenger has dementia.

12. The vehicle control method of claim 11, wherein determining whether to perform autonomous driving includes:
turning off an autonomous driving function in response to determining that the passenger has dementia; and
outputting a warning indicating that it is impossible to drive.

13. The vehicle control method of claim 11, further comprising:
outputting a virtual sound based on vehicle environment information in response to determining that the predetermined autonomous driving level is approved.

14. The vehicle control method of claim 13, wherein outputting the virtual sound includes:
tuning the virtual sound in conjunction with the biometric information.

15. The vehicle control method of claim 11, wherein determining whether to perform autonomous driving includes:
outputting a virtual sound based on an emotional state of the passenger in response to determining that the passenger does not have dementia.

16. The vehicle control method of claim 11, wherein determining whether the passenger has dementia includes:
comparing an electroencephalogram (EEG) measurement value with a predetermined reference EEG value, when a peak is detected at a specified frequency of EEG;
determining whether the predetermined autonomous driving level is less than or equal to a reference autonomous driving level, when a difference between the EEG measurement value and the reference EEG value is greater than or equal to a threshold; and
determining not to approve autonomous driving in response to determining that the predetermined autonomous driving level is less than or equal to the reference autonomous driving level.

17. The vehicle control method of claim 16, wherein determining whether the passenger has dementia includes:
obtaining a physical parameter and an emotional parameter based on the voice interaction, when the difference between the EEG measurement value and the reference EEG value is not greater than or equal to the threshold;
analyzing a linguistic characteristic of the passenger based on the physical parameter and the emotional parameter;
comparing the linguistic characteristic of the passenger with a linguistic characteristic of a dementia patient; and
determining not to approve the reference autonomous driving level or less, when a similarity between the linguistic characteristic of the passenger and the linguistic characteristic of the dementia patient is greater than or equal to a predetermined reference value.

18. The vehicle control method of claim 16, wherein determining whether the passenger has dementia includes:
detecting an arrhythmia pattern based on a heart rate signal, when the peak is not detected at the specific frequency of the EEG;
detecting breathing instability based on a breathing rate and a breathing sound;
comparing the EEG measurement value with the reference EEG value, when the arrhythmia pattern and the breathing instability are detected; and
determining whether to approve the reference autonomous driving level based on the result of comparing the EEG measurement value with the reference EEG value.

19. The vehicle control method of claim 11, wherein the biometric information includes at least one of an EEG, heart rate, blood pressure, breathing sound, a body temperature, or a combination thereof.
